(19) United States
(12) Patent Application Publication
(10) Pub. No.: US 2020/0253898 A1
(43) Pub. Date: Aug. 13, 2020

(54) MATERIALS AND METHODS FOR THE CONTROL OF BIOFILM

(71) Applicant: INNOVATION TECHNOLOGIES, INC., Lawrenceville, GA (US)

(72) Inventors: Carolyn L. TWOMEY, Lawrenceville, GA (US); Gareth CLARKE, Lawrenceville, GA (US); Samuel ZAIDSPINER, Lawrenceville, GA (US)

(21) Appl. No.: 15/763,219

(22) PCT Filed: Oct. 26, 2017
(86) PCT No.: PCT/US2017/058510

§ 371 Date: Mar. 26, 2018

Related U.S. Application Data
(60) Provisional application No. 62/413,116, filed on Oct. 26, 2016.

(51) Int. Cl. A61K 31/155; A61K 9/107; A61K 9/00; A61K 35/741; A61P 31/04; A61P 35/00

(52) U.S. Cl. CPC A61K 31/155; A61K 9/107; A61K 9/0019; A61P 35/00; A61K 35/741; A61P 31/04; A61K 9/0073

(57) ABSTRACT

The subject invention provides materials and methods for preventing, inhibiting or reducing biofilm formation and biofilm infections in subjects. The materials and methods utilize chlorhexidine, which has been found to be surprisingly non-toxic. The lack of toxicity facilitates the use of chlorhexidine in contexts that were not previously thought to be possible.

### CROSS-REFERENCE TO A RELATED APPLICATION

This application claims the priority benefit of U.S. Provisional Application Ser. No. 62/413,116, filed Oct. 26, 016 which is incorporated herein by reference in its entirety.

### BACKGROUND OF THE INVENTION

The previous understanding of germ theory directs bacterial treatments to bacteria in a free-floating (planktonic) state. Antibiotics, which are the main tools in treating infections, are based on the efficiency of microbial killing studied in free-floating (planktonic) state, functioning as a single cell. Quantification of antibiotic efficacy is done in, for example, traditional Minimum Inhibitory Concentration (MIC) assays. However, certain human (and other animal) infections are now understood to be due to the coordinated, en masse behavior of entire microbial colonies. These colonies are often composed of microbes working together in a biofilm state. A component of the biofilm surrounds and protects the entire colony from antibiotics and attacks by an intact immune system.

Biofilms are initiated when free-floating, planktonic bacteria anchor to biologic or inert surfaces such as indwelling medical devices. The attached bacteria multiply and progress from a state of monolayer to a microcolony and then to a critical mass, at which bacterial crosstalk occurs, triggering a phenomenon known as quorum sensing that leads to the biofilm phenotype. Quorum sensing turns on biofilm-producing genes not expressed or produced in non-sessile bacteria. The bacteria respond collectively to express factors that are specific to the biofilm phenotype, which lead to the secretion of an exopolysaccharide (EPS) matrix definitive of biofilm. This biofilm phenotype is characterized morphologically by the formation of microbial towers, which are composed of layers of embedded, live bacteria with intervening water channels. Under the right environmental conditions, free-floating bacteria are released from the biofilms, and the cycle is continued at other surfaces.

Pathogenic biofilms behave completely differently from the very same bacteria in free-floating, non-biofilm producing form. Due to different genomic expression, biofilm-related infections have a different clinical course and antibiotic response than planktonic-type infections. Moreover, treating biofilm associated infections “the same” as planktonic infections creates antibiotic-resistant bacteria because the EPS matrix generated by the colony gives the colony 1000-fold resistance against antibiotics that would ordinarily kill these microbes if in free-floating form.

When encased in biofilms in the human body, bacteria are a thousand times less susceptible to antibiotics, making certain infections, such as pneumonia difficult to treat and potentially lethal.

Because antibiotics fail to eradicate these EPS-protected microbial communities, use of antibiotics can compound the problem because antibiotics select for and perpetuate increasingly antibiotic-resistant bacteria. These bacteria include methicillin-resistant Staphylococcus aureus (MRSA), the world's leading cause of nosocomial infection, and a bacterium now widespread in the community at large.

MRSA infection is caused by Staphylococcus aureus bacteria—often called “staph.” Strains of staph that were resistant to the broad-spectrum antibiotics first emerged in hospitals. These antibiotics include methicillin and other more common antibiotics such as oxacillin, penicillin, and amoxicillin. MRSA was one of the first germs to be resistant to all but the most powerful drugs.

Staph bacteria are generally harmless unless they enter the body through a cut or other wound. In older adults and people who are ill or have weakened immune systems, ordinary staph infections can cause serious illness. Decades ago staph infections, including MRSA, occurred most frequently among persons in hospitals and healthcare facilities, such as nursing homes and dialysis centers, who have weakened immune systems; however, in the 1990s, a type of MRSA began appearing in the wider community. Today, that form of staph, known as community-associated MRSA, or CA-MRSA, is responsible for many serious skin and soft tissue infections and for a serious form of pneumonia. If not treated properly, MRSA infection can be fatal.

MRSA infections in the community are usually manifested as skin infections, such as pimples and boils. These CA-MRSA infections can occur in otherwise healthy people, and commonly occur among athletes who share equipment or personal items including towels and razors. There have been a number of reported outbreaks of CA-MRSA affecting high school and professional athletic teams. The susceptibility of athletes to these infections is aided by the fact that MRSA grows very rapidly in warm, moist areas such as gyms and gym locker rooms. Common cuts and abrasions such as those frequently occurring in football and baseball now pose significant threats due to the possibility of an MRSA infection. Additionally, recent research has suggested that 30-50% of the population carries MRSA colonies on their bodies all the time, helping to facilitate the spread of infection.

Despite the domestic and global ramifications, modern medicine has few treatments for pathogenic biofilm-associated infections. Furthermore, the solution to this problem is not merely the development of another new antibiotic because, in order to avoid perpetuation of antibiotic-resistant bacteria, such treatments must have broad-spectrum as well as anti-biofilm activity. This is reflected time and time again in real patients, for whom even repeat, extended courses of antibiotics “proven” effective in MIC tests are often unsuccessful.

Vancomycin is one of the few antibiotics still effective against hospital strains of MRSA, although the drug is no longer effective in every case. Several drugs continue to work against CA-MRSA, but CA-MRSA is a rapidly evolving bacterium, and it may be a matter of time before it, too, becomes resistant to most antibiotics.

Biofilms have broad-ranging clinical relevance in all areas of medicine. Bacterial biofilms such as those commonly associated with Pseudomonas and Staphylococcus are known to be a cause of intractable infection as well as chronic low-grade inflammation. The bacterial colonies in bacterial biofilms appear to be very resistant to the hosts' natural defenses as well as antibiotic treatments. Biofilms colonize virtually any surface in or on the human body to which these colonies can adhere. They often colonize biomaterials such as urinary catheters, transcutaneous intravenous lines and prosthetic heart valves.

In the living environments, biofilm can cause slime, clogging, and malodor in drains, pipes, etc. In some cases, biofilm formed on the surface of equipment necessary for food processing causes food poisoning or the like, due to adhesion of microorganisms to food after processed.

Attacking, dissolving or otherwise weakening the bacterial biofilm matrix, interrupting the quorum mechanisms maintaining the bacterial community, as well as upregulating local host innate immunity could cure what would otherwise become incurable chronic infection or chronic biofilm-associated inflammatory disease. Penetration or dispersion of the bacterial biofilm “armor” is critical in fighting biofilm-induced chronic inflammation, particularly those involving antibiotic-resistant bacteria.

Not only are bacteria in biofilm state robustly resistant to antibiotics, they are also resistant to other anti-bacterials and biocides, such as alcohols, acids and iodine solutions. In fact, today's antibiotics clearly and repeatedly demonstrate profound failure to treat biofilm-associated infection. Moreover, there are no well-known or proven anti-biofilm treatments per se. Attempts to treat infections presumed secondary to pathogenic biofilm formation include repeated and prolonged antibiotic therapy, physical removal of the biofilm (i.e., surgery or debridement) and topical sterilizers such as alcohol based foams or gels used for hand cleansing. Not only do these treatments fail to restore normal physiology, they disrupt the homeostasis of innate immunity—antibiotics breed increasingly resistant bacteria, surgery or debridement results in anatomic wounding which creates another potential site for infection, and topical disinfectants may encourage development and growth of pathogenic biofilms by eradicating normal commensals as well as pathogens. Therefore, developing methods and materials of inducing biofilm dissociation and/or prevention of biofilm secretion is an area of increasing research.

It would also be desirable for a treatment to be applied directly to the areas affected by pathogenic biofilms, including surfaces such as human mucosa and keratinized and non-keratinized epithelium and indwelling medical devices. Such administration techniques would circumvent systemic toxicity because they are by definition administered via localized (skin medicament, nasal spray, oral inhaler or nebulizer, ocular drop, oral troche, et cetera) delivery systems. Also desirable would be for treatments to be inexpensive and safe, for example, if treatments were to be comprised of natural, generally regarded as safe (GRAS) derivative/non-pharmaceutical ingredients. Lastly, it would be useful if anti-biofilm compositions could be applied to inert surfaces (i.e., hospital equipment, airplane tray tables, school desks) to limit the spread/presence of pathogenic biofilms in the hospital/clinical environment as well as in the community at large.

Chlorhexidine is a chemical antiseptic that is often used as an ingredient in mouthwash designed to kill dental plaque and other oral bacteria. Chlorhexidine also has non-dental applications. For example, it is used for general skin cleansing, as a surgical scrub, and as a pre-operative skin preparation. Chlorhexidine is typically used in the form of acetate, gluconate, or hydrochloride, either alone or in combination with other antiseptics such as cetrimide.

The use of chlorhexidine in wound irrigation applications has been previously described. See, for example, U.S. Published Application No. 2011-0288507A and U.S. Published Application No. 2011-0097372A, both of which are incorporated herein, by reference, in their entireties.

### BRIEF SUMMARY OF THE INVENTION

The current invention provides materials and methods for preventing, treating, or disrupting a biofilm-associated infection by administering a disinfectant composition comprising chlorhexidine, either directly or indirectly, to the site of the infection, or potential infection.

The current invention also provides materials and methods for disrupting, dissociating, penetrating biofilm and/or preventing biofilm secretion by administering a disinfectant composition comprising chlorhexidine, either directly or indirectly to the site of the biofilm, or potential formation of biofilm.

In preferred embodiments, the anti-biofilm composition is sterile and is administered directly to the biofilm at a pressure sufficient to disrupt the biofilm.

The compositions of the subject invention can be delivered to the affected tissues by direct application, significantly increasing efficacy. In preferred embodiments, the chlorhexidine solution is administered to the biofilm at a pressure of at least 7 psi, more preferably 10 psi or greater, and most preferably at 12 psi or greater. In preferred embodiments the pressure is less than 25 psi and preferably less than 20 psi.

Advantageously, it has been found that chlorhexidine-containing solutions can be administered to a subject according to the current invention without causing hemolysis or other negative effects on the blood, blood cells, or vascular system. Furthermore, when administered according to the procedures of the subject invention, the chlorhexidine-containing solutions of the subject invention do not result in deleterious absorption of chlorhexidine, systemic toxicity, or fibrosis. Furthermore, the compositions of the subject invention can be applied to tissue of the nervous system, including tissue of the central nervous system (CNS), without causing harmful effects. Finally, in accordance with the subject invention the chlorhexidine-containing solution can be applied in the presence of articular tissue/chondrocytes without toxicity.

Based on these findings it is now possible to utilize chlorhexidine-containing solutions in novel and advantageous ways, as described herein, to effectively treat and/or prevent, or disrupt biofilm and/or biofilm-associated infections in a wide range of tissues and locations in a subject.

Advantageously, the anti-biofilm compositions of the subject invention are useful for eliminating biofilm having, or associated with, drug resistance, including MRSA-formed biofilm. Furthermore, microbes do not readily acquire resistance to the treatments of the subject invention.

In a preferred embodiment, the active agent applied according to the subject invention is chlorhexidine gluconate, preferably at a concentration of about 1.0% or less, more preferably at about 0.1% or less, more preferably less than 0.08% and even more preferably at about 0.05% or less, and for some uses at 0.02% or less. Chlorhexidine in solution in sterile water can be used according to the current invention.

In certain embodiments, the administration of the chlorhexidine-containing solution is followed by a rinse with, for example, saline. Data demonstrate there is minimal removal of CHG bound to the tissue or bacterial organism using a saline rinse. In other embodiments, no such rinse is applied. In certain embodiments, such as in the case of surgeries and/or irrigating a body cavity, the administration of chlorhexidine can be followed by suction or alternative methods of removal such as blotting with a sterile ray tech or sponge. The suction may be applied, for example, after allowing for 30 seconds, 1 minute, 2 minutes, 5 minutes or more after the chlorhexidine is administered.

The aqueous solution, or other material, containing chlorhexidine may have other components including, for example, pH modifiers, buffers, local anesthetic agents, agents that promote wound healing, agents that help degrade biofilm, agents that stop bleeding and/or promote clot formation, and other therapeutic and non-therapeutic components.

In one embodiment, the composition “consists essentially” of an aqueous solution of CHG. which means that the solution contains no other active agent, other than chlorhexidine gluconate, that materially changes the ability of the solution to control biofilm growth.

The disinfectant composition of the current invention can be used in a variety of applications directed at preventing and/or treating biofilm related infections. Treatment can be applied at, for example, a surgical site, a surgical incision on the skin, the blood, the urogenital tract, an implant, a joint, the respiratory tract, an intraperitoneal site, an ocular site, the colon, the sinuses, an intra-articular site, a mediastinal site, a healing tissue site, intracranial, or a cerebrospinal site, or other nervous system tissue.

Also based on anatomic area of involvement, the present invention may use a two or more step application process, e.g., localized application of a first composition to decrease pathological biofilms, followed by application of a second composition to promote restoration of normal commensal bacterial homeostasis. A step of applying an antibiotic can also be used.

The compositions of this invention can also be applied to inert surfaces (e.g. hospital equipment, airplane tray tables, school desks, tubings, and pipes) to limit the spread/presence of pathogenic biofilms in the environment as well as in the community at large.

In one embodiment, the subject invention provides methods for prevention and/or treatment of diseases caused by, or associated with, biofilms or antibiotic resistant microbes. In one embodiment, the method comprises administering, to a subject in need of such treatment, an effective amount of a composition of the subject invention.

In certain embodiments, the chlorhexidine treatment is used to treat a subject who has been diagnosed as having a biofilm infection and/or a subject who has been diagnosed as being at risk for acquiring a biofilm infection.

The current invention also provides kits and trays comprising the anti-biofilm composition and apparatuses or devices for administration of the anti-biofilm composition to the subject. In preferred embodiments the composition, the kits and the trays are sterile.

### DETAILED DESCRIPTION OF THE INVENTION

The present invention provides materials and methods for disrupting and/or inhibiting the growth of biofilm.

In preferred embodiments, the current invention provides materials and methods for preventing and/or reducing the development of a biofilm related infection or treating an existing biofilm related infection at a site in a subject. The subject may be, for example, a human or other animal. The treatment can also be applied to inanimate surfaces.

The current invention also provides materials and methods for eliminating, disrupting, dissociating, penetrating biofilm and/or preventing biofilm secretion by administering a disinfectant composition comprising chlorhexidine, either directly or indirectly to the site of the biofilm, or potential biofilm. In preferred embodiments, the disinfectant composition is sterile.

The compositions of the subject invention can be delivered to the affected tissues (or other site) by direct application, significantly increasing efficacy. The composition can be applied directly to an area affected by a biofilm, including surfaces such as human mucosa and keratinized and non-keratinized epithelium. It may also be applied directly to another medical device such as, but not limited to, surgical mesh, vascular grafts, breast implants, or other implantable medical devices.

Examples of such locally directed therapies include skin medicaments, nasal sprays and washes, ear drops, rectal administration, oral inhalers and nebulizers, ocular drops, contact lenses, contact lens solutions, oral troches, dentifrices such as mouthwash, toothpaste, floss, and periodontal treatment. In each case, the composition of the present invention is administered via a vehicle whose composition is physiologically appropriate based on the area of anatomic administration.

In certain embodiments, the chlorhexidine treatment is used to treat a subject who has been diagnosed as having a biofilm infection and/or a subject who has been diagnosed as being at risk for acquiring a biofilm infection.

Also based on anatomic area of involvement, the present invention may use a two or more step application process, e.g., localized application of a first composition to decrease pathological biofilms, followed by application of a second composition to promote restoration of normal commensal bacterial homeostasis and/or an antibiotic agent effect against, for example, microbes in the planktonic state.

Chlorhexidine-containing compositions can be administered to a subject according to the current invention without causing hemolysis or other harmful effects on the blood, blood cells, or vascular system. Furthermore, when administered according to the procedures of the subject invention, the chlorhexidine-containing solutions of the subject invention do not result in deleterious absorption of chlorhexidine, systemic toxicity, or fibrosis. Additionally, the compositions of the subject invention can be applied to tissue of the nervous system, including tissue of the central nervous system (CNS), without causing harmful effects.

Based on these findings it is now possible to utilize chlorhexidine-containing compositions in novel and advantageous ways, as described herein, to effectively treat, disrupt, and/or prevent biofilm related infections in a wide range of tissues and locations in or on a subject.

In one embodiment, the subject invention provides a method for preventing, inhibiting, or reducing a biofilm formation or a biofilm infection at a site in a subject, wherein said method comprising administering to the site an aqueous solution that comprises chlorhexidine at a concentration of 1% or less (preferably 0.05% or less), and wherein the site is selected from a) blood, b) a urogenital tract, c) a respiratory tract, d) an intraperitoneal site, e) an ocular site, f) the colon, g) the sinuses, h) an intra-articular site, i) a mediastinal site, and j) a cerebrospinal site.

Advantageously, the anti-biofilm compositions of the subject invention are useful for eliminating biofilm or reducing the formation of biofilm against drug resistance, including MRSA-formed biofilm.

In one embodiment, the method of the subject invention comprises the steps of:

(a) providing a sterile composition comprising an active agent comprising chlorhexidine at a concentration of about 1% or less, 0.08% or less, 0.05% or less, or 0.02% or less, and

(b) administering the sterile composition, directly or indirectly, to the site in the subject.

In a specific embodiment, the patient is first diagnosed with a biofilm infection. In a further specific embodiment, the infection has previously been treated with a different anti-microbial agent, such as an antibiotic. In a further specific embodiment, the infection has previously been treated with a different anti-microbial agent and the infection has been determined to be resistant to the previously-used antimicrobial agent. The previously used antibiotic may be, for example, methicillin, vancomycin, oxacillin, penicillin, and amoxicillin.

The site to which the chlorhexidine is applied can be any site that is at a risk of developing an biofilm-associated infection or has an existing infection that is associated with the formation of biofilm. Non-limiting examples of sites that are appropriate for the practice of the method of the current invention include surgical sites, surgical incisions on the skin, the blood, the urogenital tract, implants, the respiratory tract, intraperitoneal sites, ocular sites, the colon, the sinuses, the nasal passage, an intra-articular site, a mediastinal site, intracranial, a cerebrospinal site or other nervous system tissue.

Advantageously, the composition of the subject invention is effective in combating infection, in particular, anti-biotic resistant infections and biofilm-associated infections, even when organic materials (including blood, tissue, and/or dirt and debris) are present.

In a further embodiment, the anti-biofilm composition of the subject invention is effective in dispersing and eliminating newly formed biofilm as well as aged biofilm such as biofilm formed for at least 1 day, 2 days, 5 days, 1 week, 2 weeks, 3 weeks, or 1 month or more.

In a specific embodiment, the anti-biofilm composition stimulates differential growth of the microorganisms in the biofilm. The differential growth can disrupt the integrity of the biofilm and leads to enhanced susceptibility of the biofilm to further treatment of the composition, which ultimately results in the removal of biofilm.

The sterile anti-biofilm composition of the current invention contains an active agent that preferably comprises (or consists of, or consists essentially of) chlorhexidine at a concentration of less than about 1%, less than 0.08%, about 0.1% or less, less than about 0.05%, less than about 0.025%, or less than about 0.02%. The chlorhexidine can be, for example, chlorhexidine gluconate (CHG), chlorhexidine acetate, chlorhexidine hydrochloride, or a combination thereof. The chlorhexidine may also be modified with, for example, a phosphate group to enhance efficacy, further reducing the likelihood of the development of resistant microbes. The disinfectant composition can further contain one or more additional active agents. In certain embodiments, the composition contains no alcohol, or less than 0.1%, 1%, 5%, 10%, 25%, or 50% alcohol.

In certain embodiments, chlorhexidine can be incorporated into an indwelling medical device itself and/or a coating that can be applied to such a device. If desired the chlorhexidine can be released over time through the use of, for example, an appropriate hydrogel or other polymer. In specific embodiments, the chlorhexidine can be released preferentially in the presence of an infection. This can be accomplished by, for example, incorporating the chlorhexidine into a material that releases the chlorhexidine when a pH change associated with the presence of the bacteria occurs.

Further embodiments of the subject invention include nasal sprays or other forms of nasal irrigation solutions to facilitate nasal irrigation to treat infections, including those caused by biofilm and/or antibiotic resistant microbes such as MRSA. In one embodiment, the invention provides a method for treating a nasal infection associated with biofilm by administering to a subject that has been diagnosed with a biofilm or MRSA nasal infection, a solution containing an anti-infective amount of chlorhexidine. In one embodiment the chlorhexidine is CHG. In another specific embodiment, the infection is a MRSA infection.

In one embodiment, the compositions of the subject invention are used to prevent or reduce the formation of biofilm in, for example, the context of surgical implants, stents, catheters, and other indwelling medical devices. The chlorhexidine-containing solutions are used to reduce the formation of biofilm in other contexts as well, including, for example, biofilm associated with sinus infections and pink eye.

In a further embodiment, the compositions of the subject invention can be used to prevent or reduce eye infections, and for the treatment of underlying inflammatory processes associated with dry eye syndrome. The sequelae of pathogenic biofilms on or near the ocular surface can result in chronic ocular low-grade inflammatory conditions, including dry eye syndrome. The subject invention provides compositions for treating the symptoms and the causes of dry eye and ‘shifting sands, syndromes. Specifically, these compositions inhibit pathogenic biofilm growth and bring about an overall anti-inflammatory effect on the ocular/adnexal surface. In a preferred embodiment, the patient is first diagnosed with dry eyes and then a chlorhexidine solution is administered to the patient thereby treating the dry eyes syndrome.

Such treatment of the ocular and adjoining surfaces improves the homeostasis between pathogenic and beneficial microflora of the ocular-adnexal area. Rebalancing or adjusting pathogenic versus nonpathogenic or even beneficial organisms improves symptoms of chronically dry, irritated, red or inflamed eyes. Additionally, other compounds such as L-theanine, Vitamin D3, prebiotic polysaccharides, and the marine organism Spirulina can be supplemented in the composition according to the subject invention to treat conditions associated with pathological biofilm.

In other embodiments, the compositions of the current invention can be used for the prevention and/or disruption of pathological biofilms and/or chronic infections present in, associated with, or leading to, various other chronic inflammatory states such as chronic rhinosinusitis; chronic periodontitis; chronic bronchitis and other states of respiratory inflammation including aspergillosis, cystic fibrosis and asthma; inflammatory otic conditions such as “swimmer's ear,” otitis externa and chronic otitis; and inflammatory skin conditions such as atopic dermatitis and eczema. The pathophysiology of these conditions is likely to involve the disruption of the normal commensal bacterial population by pathogenic species and pathogenic biofilm formation. The subject invention improves symptoms associated with these conditions and the underlying inflammatory state.

Other uses include administering chlorhexidine in the context of breast implants or collagen implants to reduce the likelihood of infection, development of biofilm and the need for follow up surgery.

Chlorhexidine solutions of the subject invention can also be used according to reduce bacteria count disinfect acupuncture needles, earrings and other piercing objects that can then be inserted into the body.

Even further, a urogenital tract irrigation system can be used to administer the sterile disinfection composition of the subject invention to the urogenital tract of a patient.

The antibiofilm composition of the subject invention can also be administered to the respiratory system of the subject.

Additionally, a cerebrospinal irrigation system can be used to administer the sterile disinfectant composition to a site in the nervous system of a subject.

In certain embodiments, the subject invention provides a method for disrupting a biofilm at a site in a subject, wherein said method comprising identifying a biofilm infection and administering to the biofilm an aqueous solution that comprises chlorhexidine at a concentration of 1% or less, and wherein the site is selected from:
a) blood,
b) a urogenital tract,
c) a respiratory tract,
d) an intraperitoneal site,
e) an ocular site,
f) the colon,
g) the sinuses,
h) an intra-articular site,
i) a mediastinal site,
j) a cerebrospinal site,
k) an intracranial site,
l) a thoracic site,
m) skin and/or soft tissue,
n) the large or small intestine,
o) a burn, and
p) an extremity site.

In certain embodiments, the active ingredient of the current invention can be combined with antibiotics. Because the administration of chlorhexidine according to the subject invention has anti-biofilm effect, it makes the underlying biofilm-associated infection susceptible to antibiotics typically ineffective in the biofilm treatment setting. The invention also allows antibiotics to be used at a lower amount, thereby decreasing toxicity as well as treatment expense because the invention “sensitizes” the underlying pathogenic micro-organisms to antibiotic antimicrobial mechanism(s).

Some ingredients common to many, but not all, embodiments of the compositions of this aspect of the invention include antibiotic compositions obtained from or associated with, natural products. These may include microbial metabolites, cellular and/or acellular fractions used singularly or in combination with viable or nonviable probiotic or other microbes, including bacteria, fungi and cyanobacteria such as Arthrospira (Spirulina) platensis, and pharmaceutical grade honey. Other ingredients that may be used in certain embodiments include, but are not limited to, prebiotic compounds such as larch or acacia gum, other hive products such as royal jelly, bee bread and propolis, green tea derivatives such as epigallocatechin gallate (EGCG) and L-theanine, other plant derivatives such as from Inula helenium, Melaleuca alternifolia and Leptospermum scoparium and water-soluble and water-insoluble Vitamin D3.

Advantageously, in preferred embodiments, ingredients of the composition of the current invention work together to inhibit biofilm formation and biofilm-associated infections while improving associated chronic inflammatory conditions through enhancement of pathogenic biofilm dispersion as well as improvement of the normal, local innate immune response.

The compositions of the subject invention can be applied directly to the involved areas, such as human mucosal, keratinized and non-keratinized epithelial surfaces. This technique reduces or eliminates systemic toxicity, because the administration is localized (skin medicament, nasal spray, oral inhaler or nebulizer, ocular drop, oral troche, et cetera).

Anti-biofilm efficacy of compositions, including the compositions of the present invention, may be assessed using the Calgary Biofilm Device, an FDA Class I approved device for the inoculation of biofilms (U.S. Pat. No. 6,599,714, herein incorporated by reference) to perform the MBEC (Minimum Biofilm Eradication Concentration) procedure or other means of assessing anti-biofilm efficacy. Other anti-microbial tests that can be employed include: the agar or disk-diffusion technique, the Kirby-Bauer test and the Minimum Inhibitory Concentration (MIC). These techniques are well known to those versed in the art and will not be recounted in detail here. Protocols may be found in “Techniques in Microbiology” by John Lammert, Pearson Education, 2007, and “Microbiology Laboratory Fundamentals and Applications” by George A. Wistreich, Pearson Education, 2003, which are incorporated by reference in their entirety.

Antibiofilm efficacy (Biofilm Inhibitory Concentration or BIC) can be compared directly against planktonic efficacy by performing the Minimum Inhibitory Concentration (MIC) test for the same anti-microbial compounds and micro-organisms being tested. Additionally, antibiofilm efficacy can be measured using a classification system similar to the manuka factor (Molan, Peter, “Method for the assay of antibacterial activity of honey”, 2005, herein incorporated by reference), except that, in this case, what is measured is the size of complete biofilm growth inhibition (biofilm inhibitory concentration, or BIC), rather than the killing diameter (“zone of inhibition”) of antimicrobial substances of compounds such as honey. This procedure will be used to develop BIC standards of the compositions against a range of bacteria as well as bacterial groups such as gram negative bacteria, methicillin sensitive and methicillin resistant Staphylococcus, et cetera.

In certain embodiments, cellular or acellular fractions or extracts of organisms or their extracellular milieu such as a biofilm derivative itself may have particular anti-biofilm and/or anti-inflammatory efficacy that may be even more effective than the source of the fraction itself.

The use of CHG in wound irrigation applications has been previously described. See, for example, U.S. Published Application No. 2011-0288507A and U.S. Published Application No. 2011-0097372A, both of which are incorporated herein, by reference, in their entireties. Those patent applications describe various uses of CHG-containing solutions. In certain embodiments, the materials and compositions of the current invention specifically exclude those uses that were described in U.S. Published Patent Application Nos. 2011-0288507A and 2011-0097372A.

The terms “about,” “approximately,” “approximate,” and “around” are used in this patent application to describe some quantitative aspects of the invention, for example, the concentration of the active agent. It should be understood that absolute accuracy is not required with respect to those aspects for the invention to operate. When these terms are used to describe a quantitative aspect of the invention the relevant aspect may be varied by up to ±10%. Thus, the terms “about,” “approximately,” “approximate,” and “around” allow for variation of the various disclosed quantitative aspects of the invention by ±1%, ±2%, ±3%, ±4%, ±5%, ±6%, ±7%, ±8%, ±9%, or up to ±10%. For example, a sterile disinfectant composition comprising about 1% active agent can contain 0.9% to 1.1% active agent.

The term “treatment” or any grammatical variation thereof (e.g., treat, treating, and treatment etc.), as used herein, includes but is not limited to, ameliorating or alleviating a symptom of a disease or condition, reducing, suppressing, inhibiting, lessening, or affecting the progression, severity, and/or scope of a condition.

The term “prevention” or any grammatical variation thereof (e.g., prevent, preventing, and prevention etc.), as used herein, includes but is not limited to, delaying the onset of symptoms, preventing relapse to a disease, increasing latency between symptomatic episodes, or a combination thereof. Prevention, as used herein, does not require the complete absence of symptoms.

The term “effective amount,” as used herein, refers to an amount that is capable of preventing, ameliorating, and/or treating a pathological condition associated with biofilm.

In one embodiment, “a subject in need of such treatment” refers to a subject who is diagnosed with a pathological condition associated with a biofilm.

Advantageously, the disinfectant composition of the subject invention is effective in combating biofilm related infection, even when organic materials (including blood, tissue, and/or dirt and debris) are present.

### Formulations

In one embodiment of the subject invention, a low concentration solution of chlorhexidine can be used to effectively prevent or treat biofilm related infections. Advantageously, it has been found that the chlorhexidine-containing solutions can be administered to a subject according to the current invention without causing hemolysis or other deleterious effects on the blood, blood cells, or vascular system. Furthermore, when administered according to the procedures of the subject invention, the chlorhexidine-containing solutions of the subject invention do not result in deleterious absorption of chlorhexidine, system toxicity, or fibrosis. Furthermore, the compositions of the subject invention can be applied to tissue of the nervous system, including tissue of the central nervous system (CNS), without causing deleterious effects.

Based on these findings it is now possible to utilize chlorhexidine-containing solutions in novel and advantageous ways, as described herein, to effectively treat and/or prevent infections including biofilm related infections in a wide range of tissues and locations in a subject.

In specific embodiments, the chlorhexidine concentration is less than about 2%, less than about 1%, or less than about 0.1%. In a further embodiment, the chlorhexidine concentration is less than about 0.05%. In even further embodiments, the chlorhexidine concentration is between 0.02% and 0.05%. Specifically exemplified herein is the use of CHG.

In a specific embodiment, the CHG used according to the subject invention has the following chemical structure:

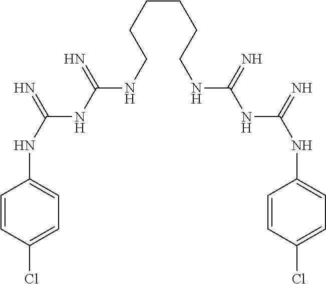 [SMILES: N=C(NCCCCCCNC(=N)NC(=N)Nc1ccc(Cl)cc1)NC(=N)Nc1ccc(Cl)cc1]

CHG

| | |
|---|---|
| Systematic | 1-[amino-[6-[amino-[amino-(4-chlorophenyl)amino- |
| (IUPAC) Name | methylidene]amino-methylidene]aminohexylimino] |
| | methyl]imino-N-(4-chlorophenyl)-methanediamine |

Chemical Data

| | |
|---|---|
| Formula | C₂₂H₃₀Cl₂N₁₀ |
| Mol. weight | 505.446 g/mol |

The pH of the disinfectant composition is preferably neutral or slightly acidic. Preferably the pH is 5.0 to 7.5. More preferably the pH is 5.5 to 7.0.

In a preferred embodiment, the administration of the disinfectant composition of the current invention to an infection site results in a reduction in the number of bacteria, other microbes or the formation of biofilm at the site when compared to either an untreated site or a site administered with saline or water that does not contain chlorhexidine. Advantageously, administration of the disinfectant composition according to the subject invention can result in effective control of a biofilm related infection without causing tissue damage.

Examples of additional active agents that can be administered to a subject in accordance with the subject invention include, but are not limited to, anti-bacterial agents, anti-viral agents, fungicidal agents, chemotherapeutic agents, topical antiseptics, anesthetic agents, oxygenated fluids and/or agents, antibiotics, diagnostic agents, homeopathic agents, probiotics, metabolites or extracts of probiotics, agents that stop bleeding, and over-the-counter medications/agents. In one embodiment, the additional agent can be an anti-microbial peptide (AMP). AMPs are well known in the art.

In certain embodiments, the additional agent is a diagnostic agent. The diagnostic agent may be, for example, an antibody, protein, or polynucleotide that binds to a target biomolecule. Any such binding may then be visualized utilizing technologies known to those skilled in the art.

For the purpose of this invention, a plain aqueous solution of the active agent comprises the active agent and/or a second agent in a solution of water that is essentially devoid of solutes that provide osmolarity to the solution, for example, a salt or a sugar. For the purpose of this invention, an isotonic solution refers to a solution having the same osmotic pressure as blood. Typically, isotonic solutions contain about 0.85% of NaCl in water.

Various embodiments of the invention can also include ocular drops, gel, ointment, cream or other vehicle of delivery of the composition appropriate to area of application, periocular lotion, gel, ointment, cream or other vehicle of delivery appropriate to the area of application, intranasal aqueous or non-aqueous spray, nasal saline rinse, skin soap, lotion, cream, emollient, and solution such as meant for contact lens cleaning and maintenance or spray.

In certain embodiments, the composition may further comprises an ingredient at a concentration (weight of the ingredient/weight of the composition) of at least about 1 μg/g, 5 μg/g, 10 μg/g, 20 μg/g, 50 μg/g, 0.1 mg/g, 0.5 mg/g, 1 mg/g, 5 mg/g, 10 mg/g, 50 mg/g, 100 mg/g, or 500 mg/g, wherein the ingredient is selected from the group consisting of extracts of microorganisms, chemical substituents, cellular or acellular components, probiotics and/or metabolites of probiotic microorganisms, honey, hive products, biosurfactants, prebiotics, plant extracts, and vitamin D.

Probiotics are micro-organisms proving beneficial in some manner to the human body. A 2001 World Health Organization symposium on probiotic micro-organisms defined these organisms as “a living micro-organism which, when it is consumed in an appropriate amount, has a positive effect on the health of its host” (World Health Organization, Joint FAO/WHO Expert Consultation on Evaluation of Health and Nutritional Properties of Probiotics in Food Including Powder Milk with Live Lactic Acid Bacteria, October 2001).

In one embodiment, the probiotic microorganism is selected from the group consisting of Aerococcus, E. coli, Bacillus, Enterococcus, Fusobacterium, Lactococcus, Leuconostoc, Melissacoccus, Micrococcus, Oenococcus, Sporolactobacillus, Streptococcus, Staphylococcus, Saccharomyces, Pediococcus, Peptostreptococcus, Proprionebacterium, and Weissella.

Biosurfactants are compounds released by microorganisms, and are generally non-toxic and biodegradable. In one embodiment, biosurfactants useful according to the subject invention are released by probiotics including non-lactic acid and lactic acid producing bacteria (LAB). In one embodiment, biosurfactants useful according to the subject invention are released by probiotics including, but not limited to, Bacteroides, Bifidobacterium, and Lactobacillus.

In additional embodiments, biosurfactants can be released by certain strains of Aerococcus, E. coli, Bacillus, Enterococcus, Fusobacterium, Lactococcus, Leuconostoc, Melissacoccus, Micrococcus, Oenococcus, Sporolactobacillus, Streptococcus, Staphylococcus, Saccharomyces, Pediococcus, Peptostreptococcus, Proprionebacterium, or Weissella.

Biosurfactants useful according to the subject invention can be glycolipids or lipoproteins. In one embodiment, the biosurfactants can be glycolipids, lipopeptides, depsipeptides, phospholipids, substituted fatty acids, lipopolysaccharides, surlactin, surfactin, visconsin, spiculisporic acid, or rhamnolipids.

Prebiotics are nondigestible, fibrous fructo- or galacto-oligosaccharides (FOS or GOS) found in many plants that are metabolized by the large intestine to form short chain fatty acids such as butyrate. These fatty acids metabolically support probiotic colonies in the intestine, as well as help generate an effective local innate immune response. Consequently, prebiotic supplementation may increase efficacy of probiotic supplementation. This combination is known as synbiotic therapy.

In certain embodiments, the invention may make use of certain prebiotics, such as locust-bean (carob) gum, in the concentration between 10 mcg-100 mg per milliliter, to augment anti-biofilm efficacy. These include fructo-oligosaccharrides (FOS), manno-ologosaccharides (MOS), galacto-oligosaccharrides (GOS), arabinogalactans and other dietary fibers, inulin, lactulose, resistant starch, isomalt, oat bran, and pectin. Larch arabinogalactan may be used and is also known as AG, Ara-6, Arabinogalactan, Arabinogalactin, dietary fiber, larch, larch gum, larch tree, larix, Mongolian Larch, Mongolian Larchwood, Soluble fiber, Stractan, Western Larch, Western Larch Arabinogalactan, Wood Gum, Wood Sugar, Larix decidua, Larix europaea, Pinus Larix, Larix occidentalis, Larix gmelinii var. gmerlinii, Larix dahurica, and Abies gmelinii. Also may be used: konjac glucomannan, also known as konjac gum, hydrolyzed konjac, hydrolyzed glucomannan, unhydrolyzed konjac, hydrolyzed glucomannan, Manna, Konjac, Konjac fiber, Devil's Tongue, and Elephant-Foot Yam. Also may be used: soluble or insoluble beta glucan, also known as the bran of cereal grains, plant cellulose, fungal components, mushroom components, seaweed components, curdlan, laminarin, chrysolaminarin, lentinan, Polysaccharide-K, lichenin, pleuran, xanthan and zymosan.

Plant extracts are known to have anti-inflammatory and anti-microbial properties. Plant extracts used in some embodiments of the invention include horseheal (Inula helenium, L. Asteraceae, elecampane), rose (Rosa damascena L., Rosaceae), lavender (Lavandula angustifolia L., Labiatae), chamomile (Matricaria recutica L., Asteraceae), orange (Rutaceae), eucalyptus (Eucalyptus globulus L., Myrtaceae), geranium (Geranium robertianum L., Geraniaceae), juniper (Juniperus communis L., Cupressaceae), citrus (Citrus sinensis L., Rutaceae), tea tree (Melaceuca alternifolia), manuka bush (Leptospermum scoparium), neem tree (Azadirachta indica, A. Juss), tea plant (Camellia sinensis) and rosemary oils (Rosmarinus officinalis L., Lamiaceae). Essential oil or water distillate of the above botanicals may be used. For instance, manuka oil at a concentration between 1-10% volume/volume (plant extract/invention) may be used.

Vitamin D has recently-discovered effects on the innate immune system besides its well-known effects on bone metabolism. Vitamin D3 induces production of anti-microbial peptides (AMPs) such as cathelicidin (LL37) on body surfaces such as the skin and eye. Vitamin D3 may be added to the formulation as an additional active ingredient. More specifically, the active form of Vitamin D may be used in an amount ranging from 1 mcg to 1 mg/ml.

### Spectrum of Activity

The composition of the subject invention is suited for biofilms which are grown under aerobic or anaerobic conditions.

Certain compositions of the subject invention can prevent or inhibit the formation of pathogenic biofilms. In addition, certain compositions of the subject invention can reduce, control or eliminate existing pathogenic biofilms.

The compositions comprising chlorhexidine can prevent or inhibit the formation of pathogenic biofilms, and/or reduce, control or eliminate existing pathogenic biofilms via a variety of mechanisms, including preventing, inhibiting, and/or disrupting the deposition, adhesion, and/or anchoring of biofilms or pathogenic microorganisms to biological or non-biological surfaces; preventing, inhibiting, and/or disrupting the secretion and/or release of extracellular factors such as exopolysaccharide (EPS) matrix; and/or preventing, inhibiting, and/or disrupting quorum-sensing mechanisms. These pathogens include aerobic and anaerobic gram-positive and gram-negative bacteria. Chlorhexidine also has activity against Candida albicans, Chlamydia trachomatis, certain fungi, and certain viruses.

Chlorhexidine is highly active against a variety of gram-positive aerobic bacteria, including Streptococcus mutants, S. pyogenes (group A β-hemolytic streptococci), S. salivarius, and S. sanguis. Chlorhexidine is active against Staphylococcus aureus, S. epidermidis, S. haemolyticus, S. hominis, and S. simulans. Chlorhexidine is active against both oxacillin-resistant (ORSA) and oxacillin-susceptible staphylococci (also known as methicillin-resistant [MRSA] or methicillin-susceptible staphylococci). Chlorhexidine is active against Enterococcus, including E. faecalis and E. faecium, and is active against both vancomycin-susceptible and vancomycin-resistant strains.

Chlorhexidine is also active against some anaerobic bacteria. Chlorhexidine is active against some strains of Bacteroides, Propionibacterium, Clostridium difficile, and Selenomonas, but is less active against Veillonella.

Chlorhexidine has activity against Candida albicans, C. dubliniensis, C. glabrata (formerly Torulopsis glabrata), C. guillermondii, C. kefyr (formerly C. pseudotropicalis), C. krusei, C. lusitaniae, and C. tropicalis (formerly C. parapsilosis). Chlorhexidine also has activity against dermatophytes, including Epidermophyton floccosum, Microsporum gypseum, M. canis, and Trichophyton mentagrophytes.

In addition to eliminate, prevent or inhibit the formation of biofilm, the sterile disinfectant composition of the subject invention can also “depathogenize” certain biofilm forming bacteria including, for example, Escherichia coli and Klebsiella aerogenes, making these bacteria less potent to cause infection.

In a preferred embodiment, the administration of the disinfectant composition of the current invention to an infection site results in a reduction of biofilm formation at the site when compared to either an untreated site or a site administered with saline or water that does not contain chlorhexidine. Advantageously, and unexpectedly administration of the disinfectant composition according to the subject invention can result in effective control of a biofilm related infection without causing tissue damage.

### Diagnosis and Treatment of Diseases Associated With Biofilm Infections

In one embodiment, the subject invention provides methods for prevention and/or treatment of diseases caused by, or associated with, biofilms. In one embodiment, the method comprises administering, to a subject in need of such treatment, an effective amount of a composition of the subject invention.

In a specific embodiment, the subject invention comprises diagnosing whether a subject has a biofilm infection, wherein the compositions of the subject invention are then administered to the subject who is diagnosed with biofilm infection. The subject may then also be monitored to access the efficacy of the treatment.

Diagnosis of biofilm infections can be accomplished by clinical techniques described in, for example, U.S. Patent Application Publication No. 2010/0285496. The location of pathogenic biofilm infection can be determined by imaging techniques such as, for example, X-ray and CT scans. In one embodiment, biofilm infection can be detected by:

a) obtaining a biological sample from a subject; and

b) measuring the presence of one or more biomarkers (e.g., exopolysaccharide, proteins, mRNA) that are associated with and/or selectively expressed by microorganisms in a biofilm state, but not in a free-floating (planktonic) state.

Thus, biofilm infection can be detected by measuring the presence of one or more biomarkers that are expressed in elevated levels by microorganisms in a biofilm state, as compared to levels in a free-floating (planktonic) state. In another embodiment, biofilm infection can be detected by the presence of bacterial extracellular polysaccharide (EPS) matrix, or chemicals contained in the EPS.

Further, species of drug resistant microbes and/or pathogenic microorganisms that form biofilm can be determined by, for example, using antibodies that recognize antigens or peptides associated with the presence of pathogenic microorganisms, or using probes that recognize nucleic acid molecules of the pathogenic microorganisms.

The term “biological sample,” as used herein, includes but is not limited to, a sample containing tissues, cells, and/or biological fluids isolated from a subject. Examples of biological samples include but, are not limited to, tissues, cells, biopsies, blood, lymph, serum, plasma, urine, cerebrospinal fluid, saliva, and tears. In certain specific embodiments, the biological samples include tears, nasal fluid, and saliva.

The presence and/or level of biomarkers useful according to the subject invention can be deteimined by techniques known in the art, such as for example, enzyme-linked immunosorbant assays (ELISA), Western blot, Northern Blot, immunological assays, immunofluorescence, and nucleic acid hybridization techniques.

Diseases Associated with Biofilm Infection

In certain embodiments, the subject invention can be used to prevent, treat, or ameliorate diseases caused by or associated with biofilm infection including, but not limited to, dermatitis, acne, chronic bronchitis, cystic fibrosis, chronic gingivitis, chronic inflammatory bowel disease, cancer, chronic eczema, chronic non-healing wounds, chronic cystitis, and medical device related inflammation such as contact lenses. The present inventors also discovered that biofilm infection causes or is associated with diseases, such as for example, chronic blepharitis and other chronic inflammatory conditions of the ocular, peri-ocular and dermatologic epithelia such as dry eye syndrome, meibomianitis and rosacea.

In one embodiment, the methods of the subject invention can be used to treat cancers, such as colon cancer, that are associated with a biofilm infection. In this embodiment, the CHG composition can be administered in conjunction with a chemotherapeutic agent and/or other cancer therapy.

In one embodiment, the subject invention can be used to prevent, treat, or ameliorate conditions in otolaryngology practice implicated by biofilms, including otitis media, chronic sinusitis, chronic tonsillitis, adenoiditis, and cochlear and middle ear implant device failures. Despite the need for improved treatment methods, prior art methods such as mechanical disruption (i.e., removal or surgical excision of the infected material) or long-term antibiotic treatment remains the treatment mainstay for chronic inflammatory states due to biofilm.

The present inventors discovered that certain ocular and peri-ocular infections result from biofilm-associated chronic inflammatory states. For example, in the ophthalmic field, the presence of biofilms has been reported on endophthalmitis after cataract surgery, on scleral buckles after retinal detachment surgery, punctal plugs, artificial nasolacrimal duct tubing and on soft contact lenses associated with keratitis. In fact, microbial contamination occurs in up to 81% of all contact lens cases, 50% of contact lenses and as 30% of all types of contact lens solutions, despite use of biocides. Infections associated with bacterial biofilm formation tend to be persistent, and the most frequently isolated organisms from biofilms are Staphylococcus aureus, S. epidermidis, and Pseudomonas aeruginosa. The ocular surfaces of dry eyes and lid margins in chronic blepharitis and contact lens wearers are colonized by significantly more bacteria and significantly more gram negative type bacteria than the typically gram positive commensal bacteria found in normal eyes.

In one embodiment, the subject invention can be used to prevent, treat, or ameliorate chronic rhinosinusitis, another example of a chronic inflammatory state associated with pathogenic biofilm formation. Pathophysiology of chronic rhinosinusitis is likely to involve the disruption of the normal commensal bacterial population by pathogens followed by pathogenic biofilm formation. Typical resulting symptoms include nasal dripping, sinus pressure, recurrent headache, post-nasal drip and cough.

In certain embodiments, the subject invention can be used to prevent, treat, or ameliorate diseases caused by or associated with biofilm infection including, but not limited to, asthma, aspergillosis, “swimmer's ear,” otitis externa, chronic otitis, atopic dermatitis, chronic rhinosinusitis, allergic rhinitis, allergic conjunctivitis, chronic bronchitis, chronic gingivitis, chronic sinusitis, and chronic periodontitis.

### Modes of Administration

The methods of the subject invention can be used in conjunction with the delivery of a chlorhexidine-containing solution by many routes. Of particular interest are: cutaneous, intra-abdominal, intracranial, intralesional, intrathoracic (during surgery), nasal, in the ear canal, as an oral bowel prep, gastric lavage, as an eye wash, periodontal, rectal, soft tissue, subcutaneous, and vaginal routes.

Chlorhexidine solutions of the subject invention can be administered using any of a wide range of currently-available delivery devices, systems, and methods. These include delivery via catheter to treat infection caused by a range of pathogenic biofilms, or potential pathogenic biofilms, including, but not limited to, urinary tract infections, bloodstream infections, intracranial infections, and joint infections. In certain embodiments the chlorhexidine solution can be administered via a syringe to treat and/or prevent spinal cord infections including, but not limited to, for example, meningitis.

The chlorhexidine solutions of the current invention can also be formulated as a spray or mist to treat appropriate sites such as chronic wounds and burns, or for nasal administration.

In a further embodiment, the subject invention provides a full-body or partial-body shower to disinfect a subject who has been, or is suspected of having been, exposed to a pathological agent such as, for example, in the context of a biological weapon.

The chlorhexidine solution of the subject invention can also be formulated for inhalation by, for example, people suffering from pneumonia or other respiratory tract infections. In a specific embodiment, the chlorhexidine solution is formulated for inhalation by cystic fibrosis (CF) patients who have developed a lung infection that associated with biofilm, or who are at risk for developing such an infection. In a specific embodiment, the subject has been diagnosed with (CF).

In a further embodiment, chlorhexidine can be incorporated into a material that can be used to disinfect skin and other bodily surfaces including, for example, the ear canal. The material may be, for example, a wipe, cloth, or swab. Preferably, the wipe, cloth, swab, or other chlorhexidine-containing material can be formulated for use even on sensitive skin such as the skin of babies or the elderly. Such wipes, cloths, swabs, and other materials can then be used in place of showers or baths for individuals who cannot readily shower or bathe. In specific embodiments, the material into which chlorhexidine has been incorporated does not include alcohol, or include less than 1% or less than 4% alcohol.

Examples of washcloths for body cleansing include U.S. Pat. Nos. 5,725,311; 5,906,278; 5,956,794; 6,029,809, and 8,221,365, all of which are incorporated herein in their entireties. In preferred embodiments, the material is impregnated with a solution comprising 1% or less of chlorhexidine and, preferably 0.05% or less. Other ingredients can be added including, for example, moisturizers.

In one embodiment of the current invention the sterile disinfectant composition can be administered to an internal surgical site (or other site of infection or potential infection) via depositing a porous material containing the active agent that releases the active agent over a period of time to the site. The presence of the active agent in and around the site can prevent and/or treat an infection. The porous material containing the active agent can be administered to a surgical site when the surgery is performed. In certain embodiments of the invention, the porous material is a disc, a sphere, or a shape designed to fit at the site.

The porous material containing the active agent can release the active agent over a period of about 1 hour to about 6 months, about 2 months to about 5 months, about 3 months to about 4 months, about 1 week to about 4 weeks, about 2 weeks to about 3 weeks, or any other peiiiiutation of these time periods.

Non-limiting examples of materials that can be used to produce the porous implants include silicate feldspar matrix, hydroxyapatite, porous titanium, or sponge. Additional examples of materials appropriate to produce sustained release implants are well known to a person of ordinary skill in the art and such materials are within the purview of the current invention. For example, Hydrogels or other such coatings that incorporate therein chlorhexidine can also be used.

In preferred embodiments of the invention, the disinfectant composition is administered to a site of healing tissue. For the purpose of this invention, a healing tissue site is an area of the tissue that suffered an injury or a disease and is recovering after the treatment for the injury or the disease. A healing tissue site can be at the surface of the skin or internal.

In certain embodiments of the current invention, the anti-biofilm composition is administered to a healing tissue site via a patch, bandage, or dressing containing the chlorhexidine; a thick viscous solution containing the chlorhexidine; a biodegradable gel; or a suture containing chlorhexidine.

Advantageously, chlorhexidine binds to healing tissues, for example, to sub-cutaneous layers of skin, to provide antimicrobial and/or healing effect. Accordingly, the sterile disinfectant composition of the current invention provides an active agent that can bind to a healing tissue to enhance healing tissue recovery, prevent infection, and/or treat an existing infection.

In additional embodiments of the invention, the sterile anti-biofilm composition can be administered to a site as a tablet taken orally, microcapsule delivery spheres, nanoparticles, targeted nanoparticles (for example, receptor mediated targeted nanoparticles), a time controlled delivery system, a frozen block of the sterile disinfectant composition, a plain aqueous solution of the active agent, an isotonic solution of the active agent, or an implantable time release delivery system. In certain embodiments, the disinfectant composition is left at the site after administration thereto.

In a further embodiment of the invention, after administration of the anti-biofilm composition of the current invention to a site or a tissue, the site or the tissue is rinsed with, for example, a sterile solution free of the active agent. Examples of solutions free of the active agent include, but are not limited to, plain water, saline, and isotonic solutions free of the active agent. The rinsing can be performed by administering the solution free of the active agent to the site and removing the resultant solution from the site or the tissue by, for example, suction. In certain embodiments, the rinsing is performed within about 1 minute to about 10 minutes, about 2 minutes to about 5 minutes, or about 3 minutes from the time of administering the sterile disinfectant composition to the site in the subject. In other embodiments, suction is performed, with or without rinsing.

Under optimal circumstances, the methods of the subject invention are utilized by trained medical technicians; however, because of the simplicity and convenience of the subject invention, they can be used to greatly enhance the effectiveness of the administration of the anti-biofilm composition regardless of the training level of the operator performing the irrigation.

The subject can be a mammal. Non-limiting examples of mammals that can be treated according to the methods of the current invention include humans, non-human primates, dogs, cats, equines, bovines, and pigs.

Following are examples that illustrate procedures for practicing the invention. These examples should not be construed as limiting.

### EXAMPLE 1—SURGICAL APPLICATIONS

In one embodiment of the current invention, the sterile anti-biofilm composition is administered to a surgical site to prevent biofilm formation or treat a biofilm related infection at the surgical site. The surgical sites may include, for example, joint replacements, abdominal surgery, brain surgery, and oral/periodontal surgery sites.

A biofilm related infection developed at the surgical site is referred to herein as “surgical site infection” or “SSI.” A surgical site is at a risk of developing an SSI from, for example, improperly handled surgical instruments or airborne infectious agents from the operating room. SSI can be treated by administering antibiotics to the patients; however, often a second surgery is required to treat the SSI. The additional surgery to treat SSI is undesirable for several reasons, for example, repeated trauma of surgery to the patient, risk of repeated infection, improper healing of the surgical site, and additional costs.

The current invention provides an easy and inexpensive alternative to the second surgery for treating an SSI. The method of the current invention as it applies to treating the SSI comprises administering to the surgical site the sterile anti-biofilm composition comprising an active agent that comprises chlorhexidine at a concentration of about 1% or less, about 0.05% or less, or about 0.02% or less.

The sterile anti-biofilm composition can be administered to the surgical site as a plain aqueous solution of the active agent. In one embodiment, after a period of time sufficient for the active agent to eliminate biofilm and/or inhibit the formation of biofilm, the surgical site can be rinsed with a sterile solution free of the active agent. Alternatively, or additionally, suction can be applied to the site. The period of time sufficient for the active agent to eliminate biofilm and/or inhibit the formation of biofilm can be about 1 minute to about 60 minutes, about 2 minutes to about 50 minutes, about 3 minutes to about 40 minutes, about 4 minutes to about 30 minutes, or about 5 minutes.

In one embodiment, a chlorhexidine solution is administered in conjunction with robotic or other minimally invasive surgeries (MIS) in order to reduce the risk of infection. In this context, tubing that delivers the chlorhexidine solution can be included with other tubes (e.g. tubes with optical components, tubes for delivery or removed or other fluids or tissue, and tubes for manipulating devices) that deliver or remove material from the surgery site, or which otherwise assist in the procedure.

Thus, in one embodiment, the subject invention provides an MIS system having, as one component, a tube through which a chlorhexidine-containing solution is discharged at a distal end of the tube. The proximal end of the tube may be configured to receive the chlorhexidine-containing solution from a reservoir that may be, for example, a bag, bottle, or other suitable container. Preferably the system is sterile. The system can have further tubes and other elements useful for conducting a MIS procedure.

The MIS system can be adapted for surgeries including, for example, coronary, vascular, prostrate, laparoscopic, spinal, and neurological.

### EXAMPLE 2—INTRAVASCULAR ADMINISTRATION

In another embodiment of the invention, the anti-biofilm composition can be administered to the blood of a subject via intravascular injection.

Preferably, the injection is intravenous. The anti-biofilm composition can be a plain aqueous solution, an isotonic solution, or other salt-containing solution that contains chlorhexidine.

In certain embodiments of the invention, an isotonic solution containing the chlorhexidine is freshly prepared before administration to the subject. For example, the isotonic solution containing the active agent can be prepared, less than 1 minute, less than 2 minutes, about 1 minute to about 30 minutes, about 5 minutes to about 20 minutes, about 10 minutes to about 15 minutes before the intravascular injection, or any other permutation of these time periods.

In certain embodiments an isotonic solution containing chlorhexidine is prepared by mixing a salt solution and chlorhexidine in an appropriate quantity of water. In certain embodiments, a volume of a plain aqueous solution of the chlorhexidine containing twice the concentration of chlorhexidine compared to the desired concentration of chlorhexidine in the final working solution is mixed with equal volume of a solution having 2× isotonicity of the isotonic solution to prepare the isotonic solution of chlorhexidine appropriate for administration into a subject's blood.

### EXAMPLE 3—UROGENITAL TRACT APPLICATIONS

In a further embodiment of the invention, the sterile anti-biofilm composition can be administered to the urogenital tract of a subject via a urogenital tract irrigation system.

A urogenital tract irrigation system refers to an apparatus useful for flushing one or more organs of the urogenital tract. Non-limiting examples of urogenital tract irrigation system include bladder irrigation systems and urethral irrigation systems.

The sterile disinfectant solution used in urogenital tract irrigation system can be, for example, a plain aqueous solution of the active agent or an isotonic solution of the active agent.

### EXAMPLE 4—INTRA-ARTICULAR APPLICATIONS AND INDWELLING DEVICES

In an even further embodiment of the current invention, the sterile anti-biofilm composition is administered to an intra-articular site via an intra-articular injection. The intra-articular sites that can be injected according to the methods of the current invention include, but are not limited to, elbow, shoulder, wrist, hip joints, knees, ankles, and intervertebral sites.

In an even further embodiment of the current invention, the anti-biofilm composition can be administered to the site of an implant or other indwelling device by incorporating the sterile disinfectant composition into or onto the implant or other devices.

For the purpose of this invention, an implant refers to a medical device designed to remain in the body for an extended period of time. The extended period of time may be, for example, more than 5 minutes, more than 1 hour, more than 12 hours, more than a day, more than a week, more than a month, and/or more than a year.

The implant may be designed to, for example, replace a missing biological structure, support a damaged biological structure, or enhance the function of an existing biological structure. Implants are man-made devices, in contrast to a transplant, which is a transplanted biomedical tissue.

The surface of implants that contact the tissue of the subject can be made of a biomedical material such as titanium, silicone, hydrogel (or other polymer) or apatite. In some cases implants contain electronics, e.g., artificial pacemakers and cochlear implants.

The active agent can be incorporated into the implant, which then releases the active agent over a period of time. The materials and time durations discussed above in connection with porous materials used to treat infections are also applicable to this embodiment of the current invention.

### EXAMPLE 5—RESPIRATORY SYSTEM APPLICATIONS

The chlorhexidine solution of the subject invention can also be formulated for inhalation by, for example, people suffering from pneumonia or other respiratory tract infections. In a specific embodiment, the chlorhexidine solution is formulated for inhalation by cystic fibrosis (CF) patients who have developed a lung infection associated with biofilm, or who are at risk for developing such an infection. In a specific embodiment, the subject has been diagnosed with (CF).

The anti-biofilm composition can be administered to the respiratory tract of a subject via inhalation of, for example, vapors, particles, and/or aerosols containing the active agent. Non-limiting examples of devices appropriate for producing vapors, particles and/or aerosols for inhalation of the active agent include inhalers and puffers. Additional examples of devices that can be used to produce inhalable vapors, particles and/or aerosols are well known to a person of ordinary skill in the art and such embodiments are within the purview of the current invention.

The compositions of the current invention can be used for the prevention and/or disruption of pathological biofilms and/or chronic infections present in, associated with, or leading to, various other chronic inflammatory states such as chronic rhinosinusitis; chronic periodontitis; chronic bronchitis and other states of respiratory inflammation including aspergillosis, cystic fibrosis and asthma; inflammatory otic conditions such as “swimmer's ear,” otitis externa and chronic otitis; and inflammatory skin conditions such as atopic dermatitis and eczema. The pathophysiology of these conditions is likely to involve the disruption of the normal commensal bacterial population by pathogenic species and pathogenic biofilm formation. The subject invention improves symptoms associated with these conditions and the underlying inflammatory state.

### EXAMPLE 6—BODY CAVITY APPLICATIONS

In one embodiment of the invention, the anti-biofilm composition is administered to a body cavity, such as an intraperitoneal site, via injection, infusion, or irrigation with the sterile anti-biofilm composition.

The anti-biofilm composition injected into the intraperitoneal site can be, for example, a plain aqueous solution of chlorhexidine, an isotonic solution, of a gel containing chlorhexidine, an emulsion, or a suspension.

### EXAMPLE 7—OCULAR APPLICATIONS

In certain other embodiments of the current invention, the sterile anti-biofilm composition is administered to an ocular site as an ophthalmic composition containing chlorhexidine. The ophthalmic composition can be, for example, a solution, suspension, spray, cream, lotion, gel, drop, soap or an ointment containing the active agent, or any other form appropriate to the site of administration. These compositions can be prepared using standard methods known to those skilled in the art.

In a specific embodiment, a chlorhexidine solution is applied to the eye in conjunction with an eye surgery procedure. The eye surgery procedure may be, for example, cataract surgery, retina surgery, lens replacement surgery, or surgery to correct traumatic damage including, but not limited to, corneal abrasion. The chlorhexidine solution may be applied before, during, or after the surgery. The chlorhexidine solution of the current invention can also be used to treat pink eye.

The concentration of the chlorhexidine may be less than 1%, preferably less than 0.16%, less than 0.05%, less than 0.02%, or even less than 0.01%. The administration of the chlorhexidine solution may be followed by a rinse with, for example, saline, but does not have to be followed by a rinse.

In one embodiment, the subject invention provides a container with a sterile chlorhexidine solution with an eye dropper contained therein, or associated therewith. The container may itself be sterile for use in a surgical setting.

In the area of ocular and adnexal tissue application, the compositions of the current invention can be used for the treatment of underlying inflammatory processes associated with dry eye syndrome. The sequelae of pathogenic biofilms on or near the ocular surface can result in chronic ocular low-grade inflammatory conditions, including dry eye syndrome. The subject invention provides compositions for treating the symptoms and the causes of dry eye syndrome. Specifically, these compositions inhibit pathogenic biofilm growth and bring about an overall anti-inflammatory effect on the ocular/adnexal surface.

Such topical treatment of the ocular and adjoining surfaces improves the homeostasis between pathogenic and beneficial microflora of the ocular-adnexal area. Rebalancing or adjusting pathogenic versus nonpathogenic or even beneficial organisms improves symptoms of chronically dry, irritated, red or inflamed eyes. Such an improvement can be brought about by an embodiment of the invention comprising a topically applied mixture of live or dead micro-organisms, and/or their extracts, as well as pharmaceutical grade honey that possesses anti-biofilm effect. Additionally, other compounds such as L-theanine, Vitamin D3, prebiotic polysaccharides, and the marine organism Spirulina can be used according to the subject invention to treat conditions associated with pathological biofilm.

The function of the ocular and adnexal microbiome is to “boost” the local innate immune system and protect the colonized surface. Cross talk between the commensal microbial flora and ocular mucosal and immune epithelial cells helps maintain ocular surface homeostasis and ocular surface health. Commensals colonizing the ocular surface include such diverse micro-organisms as Staphylococci, Corynebacterium, Streptococcus and Proprionibacterium. This microbiome remains relatively stable unless disturbed. However, there are many common situations which are likely to affect healthy ocular and peri-ocular microbiome balance—antibiotics and other medications, contact lenses, blepharitis, meibomian gland dysfunction, ocular rosacea or other causes of chronically irritated and/or dry eyes. When normal ocular and peri-ocular micro-organism populations are disturbed by any number of possible, common causes, ocular surface irritation, inflammation and discomfort result.

Application topically as described herein results in decreased inflammation of the ocular surface and surrounding areas. Since the many disparate causes of dry eye disease are united by the same immunopathogenesis of chronic inflammation, the invention may be used by the general public at large for symptomatic improvement of chronically dry, red, irritated and/or inflamed eyes.

### EXAMPLE 8—USE FOR CHRONIC WOUNDS AND BURNS

In additional embodiments, the chlorhexidine compositions of the current invention can be used for the treatment of biofilm related infection including acute and/or chronic wounds and burns. In this context, chlorhexidine can be incorporated into dressings or formulated into pastes or mists that do not cause discomfort upon application to the chronic wound or burn site.

### EXAMPLE 9—SUB-DERMAL APPLICATIONS

In a further embodiment, the chlorhexidine-containing compositions can be injected to treat sub-dermal infections such as might occur at the site of a breast implant. Advantageously, such infections can be treated according to the subject invention without the need for a further invasive procedure.

In accordance with the subject invention it has been found that chlorhexidine advantageously binds to subcutaneous tissue. Repeated application increases the chlorhexidine bound to tissue thereby creating a cumulative effect that facilitates the establishment of a barrier layer of protection against infection. In specific embodiments, chlorhexidine is applied repeatedly, or continuously, to achieve enhanced protection against infection via the establishment of an antimicrobial layer.

### EXAMPLE 10—PIERCINGS AND ACUPUNCTURE

The compositions according to the subject invention can also be incorporated into, or applied to, ear rings and other body piercing items, and acupuncture needles to reduce the incidence of infection associated with body piercings and/or acupuncture.

### EXAMPLE 11—ORAL ADMINISTRATION

In a further embodiment, the chlorhexidine-containing compositions of the subject invention can be formulated for oral delivery for treatment of sore throats as well as digestive tract maladies. In this context, the compositions of the subject invention can be used to treat the flu or other viruses as well as food poisoning and bacteria associated with ulcers and digestive tract inflammation.

### EXAMPLE 12—TREATMENT OF NASAL INFECTIONS

In further embodiments of the current invention, the sterile anti-biofilm composition is administered to the sinuses via a nasal irrigation system, a nasal swab, a nasal lavage, a nasal douche, or a neti pot. A nasal irrigation system is designed to rinse sinuses and flush out clogged nasal passages using a solution, for example, a salt solution, a plain aqueous solution, or an isotonic solution of the active agent. Additional embodiments of nasal irrigation systems are well known to a person of ordinary skill in the art and such embodiments are within the purview of the current invention.

Solutions may be prepared with or without preservatives and/or anti-oxidants and/or viscosity enhancers. Solutions may be filtered through 0.2 micron filters (Millipore) into sterile 10 mis disposable containers. The solutions may or may not be packaged with nasal rinse bottles of appropriate volume to reach appropriate tonicity such that final solution when mixed with 250 mis water is isotonic.

### EXAMPLE 13—NERVOUS SYSTEM APPLICATIONS

In certain embodiments of the current invention, the sterile anti-biofilm composition is administered to a cerebrospinal site via cerebrospinal injection or cerebrospinal irrigation.

### EXAMPLE 14—SUTURES

Additionally, sutures containing chlorhexidine may be used to stitch a surgical incision or a wound of a subject. The sutures can then release the chlorhexidine to the site of administration over a period of time. Chlorhexidine can also be added, according to the subject invention, to surgical glues and liquid bandages.

### EXAMPLE 15—DENTAL AND PERIODONTAL USE

In certain embodiments of the current invention, the sterile anti-biofilm composition may be formulated for dental and periodontal use. The chlorhexidine-containing composition may be formulated in toothpaste, or modified so that it could be used as a coating for dental floss, incorporated into a mouthwash, gum or lozenge.

### EXAMPLE 16—KITS AND TRAYS

A further embodiment of the current invention provides kits comprising the sterile anti-biofilm composition and apparatuses or devices for administration of the sterile anti-biofilm composition to the site of the subject.

The apparatuses and the devices for the administration of the sterile disinfectant composition to the site of the subject include, but are not limited to, a bottle for administering the plain aqueous solution of the active agent or the isotonic solution of the active agent to the site, a transdermal patch, a porous material, a sponge, sutures, a urogenital tract irrigation system, an implant, a vapor inhalation device, a nasal irrigation system, a nasal lavage, a nasal douche, a neti pot, an injection system, or a cerebrospinal irrigation system. This can also be achieved via the port on minimally invasive surgery trocars and other such devices

For the purpose of the current invention, an injection system can comprise a syringe and a needle and/or a catheter. The size of the needle and the syringe depend on the site to which the sterile disinfectant composition is administered. A person of ordinary skill in the art can determine the appropriate size of the syringe and the needle in a particular situation.

Non-limiting examples of the kits and trays according to the current invention include, a plain aqueous solution of the active agent, an isotonic solution of the active agent, a plain aqueous solution of the active agent at a 2× concentration of the active agent compared to the final working solution and a solution free of active agent having 2× isotonicity, the active agent in a solid form and sterile water or sterile isotonic solution, a transdermal patch containing the active agent, a porous material containing the active agent, a sponge containing the active agent, a thick viscous solution containing the active agent, a mist spray containing the active agent, sutures containing the active agent, a urogenital tract irrigation system and a sterile disinfectant composition, an implant containing the active agent, a vapor inhalation device and a sterile disinfectant composition, an aerosol inhalation device and a sterile disinfectant composition, an ophthalmic emulsion containing the active agent, an ophthalmic solution containing the active agent, an ophthalmic suspension containing the active agent, an ophthalmic ointment containing the active agent, a nasal irrigation system and a sterile anti-biofilm composition, a nasal lavage and a sterile anti-biofilm composition, a nasal douche and a sterile anti-biofilm composition, a neti pot and a sterile anti-biofilm composition, an injection and a sterile anti-biofilm composition, or a cerebrospinal irrigation system and a sterile anti-biofilm composition.

The kits and trays (including custom packs) can be used to practice the methods of the current invention. For example, a user can use a kit comprising a plain aqueous solution of the active agent or the isotonic solution of the active agent by administering the solution of the active agent to the site of the subject. Similarly, a user can mix equal amounts of the plain aqueous solution of the active agent at a 2× concentration and the solution free of active agent having 2× isotonicity to prepare a working isotonic solution of the active agent. A user can also dissolve the active agent in the solid form in sterile water or sterile isotonic solution to prepare a working isotonic solution of the active agent.

### EXAMPLE 17—ENVIRONMENTAL USE

A further embodiment of the current invention provides the environmental use of the anti-biofilm composition which may be formulated to an anti-biofilm spray for cleansing of inanimate surfaces that may be exposed to pathogenic biofilm colonization. The anti-biofilm compositions may be packaged in hand-pump room spray containers; each pump may dispense an aerosol volume equivalent to 1 ml of solution. This particular form may be left on the area applied and does not require washing.

It should be understood that the examples and embodiments described herein are for illustrative purposes only and that various modifications or changes in light thereof will be suggested to persons skilled in the art and are to be included within the spirit and purview of this application and the scope of the appended claims.

We claim:

1. A method for disrupting a biofilm at a site in a subject, wherein said method comprising identifying a biofilm infection and administering to the biofilm an aqueous solution that comprises chlorhexidine at a concentration of 1% or less, and wherein the site is selected from
a) blood,
b) a urogenital tract,
c) a respiratory tract,
d) an intraperitoneal site,
e) an ocular site,
f) the colon,
g) the sinuses,
h) an intra-articular site,
i) a mediastinal site,
j) a cerebrospinal site,
k) an intracranial site,
l) a thoracic site,
m) skin and/or soft tissue,
n) the large or small intestine,
o) a burn, and
p) an extremity site.

2. The method of claim 1, wherein the concentration of chlorhexidine is about 0.05% or less.

3. The method of claim 1, wherein the chlorhexidine is chlorhexidinc gluconate.

4. The method of claim 1, wherein the composition further comprises a second agent that is selected from anti-bacterial agents, anti-viral agents, fungicidal agents, chemotherapy agents, anesthetic agents, agents that reduce bleeding, and diagnostic agents.

5. The method of claim 1, further comprising applying suction to the site.

6. The method of claim 1, wherein chlorhexidine is administered to the site via a sustained release material containing the chlorhexidine.

7. The method of claim 1, wherein the composition is administered to the blood via intravenous injection.

8. The method of claim 1, wherein the method treats a chronic inflammatory condition.

9. The method of claim 1, wherein the composition is administered to the respiratory tract via inhalation of vapor and/or an aerosol.

10. The method of claim 1, wherein the composition is administered to the ocular site as an emulsion, solution, suspension, or ointment.

11. The method of claim 1, wherein the composition is administered with a biosurfactant.

12. The method of claim 1, wherein the composition is administered to the intra-articular site via an intra-articular injection.

13. The method of claim 1, wherein the composition is administered to the cerebrospinal site via a cerebrospinal injection or a cerebrospinal irrigation system.

14. The method of claim 1, wherein the composition is administered as a tablet taken orally, microcapsule spheres, nanoparticles, a time controlled delivery system, a frozen block, a plain aqueous solution, an isotonic solution, or an implantable time release delivery system.

15. The method, according to claim 1, wherein the subject is diagnosed with a biofilm infection.

16. The method, according to claim 1, wherein the solution is applied to the biofilm at a pressure of at least 7 psi.

17. The method, according to claim 1, used to treat an infection caused by an antibiotic resistant microorganism.

18. The method, according to claim 1, further comprising the administration of an antibiotic.

19. The method, according to claim 1, further comprising the administration of a prebiotic or a probiotic.

20. The method, according to claim 1, used to treat, or inhibit the progression of, colon cancer.
